(19) United States
(12) Patent Application Publication
(10) Pub. No.: US 2002/0107560 A1
(43) Pub. Date: Aug. 8, 2002

(54) Controlled detachment stents

(75) Inventor: Jacob Richter, Ramat Hasharon (IL)

Correspondence Address: KENYON & KENYON, Suite 700, 1500 K Street, N.W., Washington, DC, 20005, US

(73) Assignee: MEDINOL, LTD., Tel Aviv (IL)

(21) Appl. No.: 10/116,159

(22) Filed: Apr. 5, 2002

Related U.S. Application Data
(63) Continuation of application No. 09/204,830, filed on Dec. 3, 1998.

(51) Int. Cl. A61F 2/06

(52) U.S. Cl. 623/1.11

(57) ABSTRACT

A stent is provided with specific “designated detachment” points or zones, such that after the stent is deployed, the stress applied on the stent will cause the stent to detach at these designated detachment points or zones. When the detachment occurs completely around the circumference of the stent, the stent separates into stent segments, each able to move with the vessel independently of the other stent segments. The components at the designated detachment zones may have a cross-sectional area sufficiently low so that the components will detach under the stress placed on the stent after implantation. Alternatively or additionally, the components at the designated detachment zones may be made of a material that is sufficiently weaker so that the components will detach under the stress placed on the stent after implantation. The stent may have a lower number of components at the designated detachment zones than in the stent segments.

### FIELD OF THE INVENTION

The invention relates generally to stents, which are endoprostheses implanted into vessels within the body, such as a blood vessels, to support and hold open the vessels, or to secure and support other endoprostheses in vessels.

### BACKGROUND OF THE INVENTION

Various stents are known in the art. Typically stents are generally tubular in shape, and are expandable from a relatively small, unexpanded diameter to a larger, expanded diameter. For implantation, the stent is typically mounted on the end of a catheter, with the stent being held on the catheter at its relatively small, unexpanded diameter. By the catheter, the unexpanded stent is directed through the lumnen to the intended implantation-site. Once the stent is at the intended implantation site, it is expanded, typically either by an internal force, for example by inflating a balloon on the inside of the stent, or by allowing the stent to self-expand, for example by removing a sleeve from around a self-expanding stent, allowing the stent to expand outwardly. In either case, the expanded stent resists the tendency of the vessel to narrow, thereby maintaining the vessel's patency.

Some examples of patents relating to stents include U.S. Pat. No. 4,733,665 to Palmaz; U.S. Pat. No. 4,800,882 and 5,282,824 to Gianturco; U.S. Pat. Nos. 4,856,516 and 5,116,365 to Hillstead; U.S. Pat. Nos. 4,886,062 and 4,969,458 to Wiktor; U.S. Pat. No. 5,019,090 to Pinchuk; U.S. Pat. No. 5,102,417 to Palmaz and Schatz; U.S. Pat. No. 5,104,404 to Wolff; U.S. Pat. No. 5,161,547 to Tower; U.S. Pat. No. 5,383,892 to Cardon et al.; U.S. Pat. No. 5,449,373 to Pinchasik et al.; and U.S. Pat. No. 5,733,303 to Israel et al.

One object of prior stent designs has been to insure that the stent has sufficient radial strength when it is expanded so that it can sufficiently support the lumen. Stents with high radial strength, however, tend also to have a higher longitudinal rigidity than the vessel in which it is implanted. When the stent has a higher longitudinal rigidity than the vessel in which it is implanted, increased trauma to the vessel may occur at the ends of the stent, due to stress concentrations on account of the mismatch in compliance between the stented and un-stented sections of the vessel.

### SUMMARY OF THE INVENTION

An object of the invention is to provide a stent that more closely matches the compliance of the vessel in which it is implanted, with relatively little or no sacrifice in radial strength, even when the stent is made very long.

In accordance with one embodiment of the invention, a stent is provided with specific “designated detachment” points, such that after the stent is deployed, and during the motion of the vessel, the stress applied on the stent will cause the stent to segment at these designated detachment points. When the designated detachment points are arranged completely around the circumference of the stent, creating a circumferential “designated detachment” zone, the detachment at the designated detachment points separates the stent into two or more separate stent segments, each able to move with the vessel independently of the other stent segments. Because each stent segment can move with the vessel independently of the other stent segments, the series of stent segments achieves greater compliance between the stented and un-stented sections of the vessel than the longer, unitary stent, and it thereby reduces stress on the vessel wall.

The stent is preferably designed such that after detachment, the ends of the stent segments created by the detachment are relatively smooth, so that they do not injure the vessel wall. Also, the stent is preferably configured such that the individual stent segments have sufficient radial strength after detachment, such that the detachment results in little or no significant reduction in the stent's resistance to compression.

The stent may be designed such that detachment occurs only after a period of time after implantation, so that the stent will already be buried under neointima at the time of detachment. Thus, the stent segments remaining after detachment will be held in place by the neointima and will not move relative to the lumen, i.e., they will not “telescope” into one another, and they will not move away from one another, creating unsupported gaps.

A variety of mechanisms may be used to accomplish the detachment. For example, the stent may be provided at certain points or zones along its length with components having a cross-sectional area sufficiently low so that the stent segments will detach preferentially under the stress placed on the stent after implantation. Alternatively or additionally, the stent may be provided at certain points or zones along its length with components made of a material that is sufficiently weaker than elsewhere in the stent so that the stent segments will detach preferentially under the stress placed on the stent after implantation. Alternatively or additionally, the stent may be designed such that it has a lower number of components, or struts, at the designated detachment zones, so that each such component bears more load than components elsewhere in the stent. These components are configured to separate under the increased loads they bear when the stent is repeatedly stressed after implantation.

The factors contributing to detachment may be applied individually or in combination. For example, the designated detachment struts may have low cross-sectional areas and also may be formed of weaker material, or the designated detachment zones may have a reduced number of components, with or without the components having low cross-sectional areas and/or being formed of weaker material.

### BRIEF DESCRIPTION OF THE DRAWINGS

FIG. 1 shows a schematic diagram of a stent, generally in the form of a cylinder, having designated detachment zones between stent segments;

FIG. 2 shows a schematic diagram of the stent of FIG. 1 after detachment, in which the stent has separated into a series of shorter stent segments;

FIG. 3 shows a flat layout of a stent pattern in which the components in the designated detachment zones have a cross-sectional area that is sufficiently low so that the stent segments will detach under the stress placed on the stent after implantation;

FIG. 4 shows a flat layout of the stent pattern of FIG. 3, after detachment has occurred at the designated detachment zones; and

FIG. 5 shows a flat layout of a stent pattern in which the stent has a lower number of components at the designated detachment zones, so that each such component bears an increased load and separates under such increased load.

### DETAILED DESCRIPTION OF THE DRAWINGS

FIG. 1 shows a schematic diagram of a stent 1, generally in the form of a cylinder. The stent 1 comprises a series of stent segments 2 separated by designated detachment zones 3. The designated detachment zones 3 comprise one or more designated detachment components or struts (see FIGS. 3 through 5).

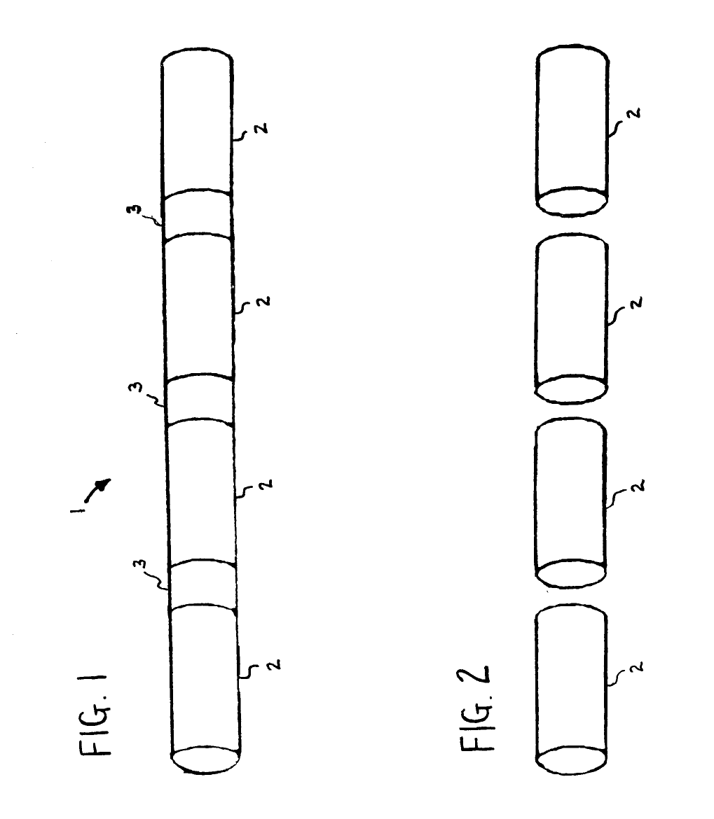

The designated detachment zones 3 are designed such that the designated detachment components or struts separate under repeated stress placed on the stent 1 after implantation. When all of the designated detachment struts around the circumference of the stent in a particular designated detachment zone 3 separate, the stent is itself separated into a series of independent stent segments 2, as shown in FIG. 2. The designated detachment zones 3 may be designed such that detachment does not occur until some time has passed after implantation, so that the stent segments 2 will already be buried under neointima at the time of detachment and therefore will not move relative to the lumen.

Persons of ordinary skill in the art will appreciate that the basic geometry of the stent segments 2 may take any suitable form, and-that the stent segments 2 may be formed of any suitable material. Examples of suitable structures for the stent segments 2 include those shown in U.S. Pat. No. 5,733,303 to Israel et al., the disclosure of which is hereby expressly incorporated by reference into this application.

FIG. 3 shows a flat layout of a stent pattern comprising stent segments 2 separated by designated detachment zones 3. In the finished stent, each stent segment 2 in this embodiment has a configuration generally corresponding to a stent configuration disclosed in U.S. Pat. No. 5,733,303. The stent segments 2 are joined to each other by the designated detachment components or struts 4 in the designated detachment zones 3.

In this embodiment, each of the designated detachment struts 4 has a reduced cross-sectional area that is sufficiently low to allow separation of the designated detachment struts 4 under the stress placed on the stent after implantation. The amount of reduction of the cross-section of the detachment struts 4 as compared to, for example, the components labeled by reference numeral 5 in the stent segments 2, may be, for example, on the order of tens of percents. For example, the detachment struts 4 may be 25% to 75% thinner or narrower than the components 5.

These designated detachment struts 4 may additionally or alternatively be made of a weaker material, in order to insure appropriate separation. The weaker material may be provided either in the stock material used to form the designated detachment struts 4, or by treating the designated detachment struts 4 (or the designated detachment zones 3) after the stent has been produced, such that the treatment weakens the material of the designated detachment struts 4.

One example of a manner of providing the designated detachment struts with weaker material is to form the entire stent of NiTi and then to treat the designated detachment struts to be Martensitic while the remaining components will be in the Austenitic phase. Another example, for example in a stent made of SST, is to anneal the components in the designated detachment zones and harden the components in the stent segments.

In addition to the reduction in cross-section, the remaining geometry of the designated detachment struts may be selected to achieve the desired results. As shown in FIG. 3, the width A of the row of designated detachment struts 4 may be narrower than the width of a corresponding row of components in the stent segment 2, for example the width B of the row of components labeled by reference numeral 5. This reduced width at the designated detachment zones 3 helps to insure detachment at the designated detachment zones 3 under longitudinal repeat bending. Also, the designated detachment struts 4 may be made sufficiently short to reduce the length of the free ends after separation, so as not to leave long, hanging ends after detachment. For example, the length of the designated detachment struts 4 is shorter than the length of the components 5.

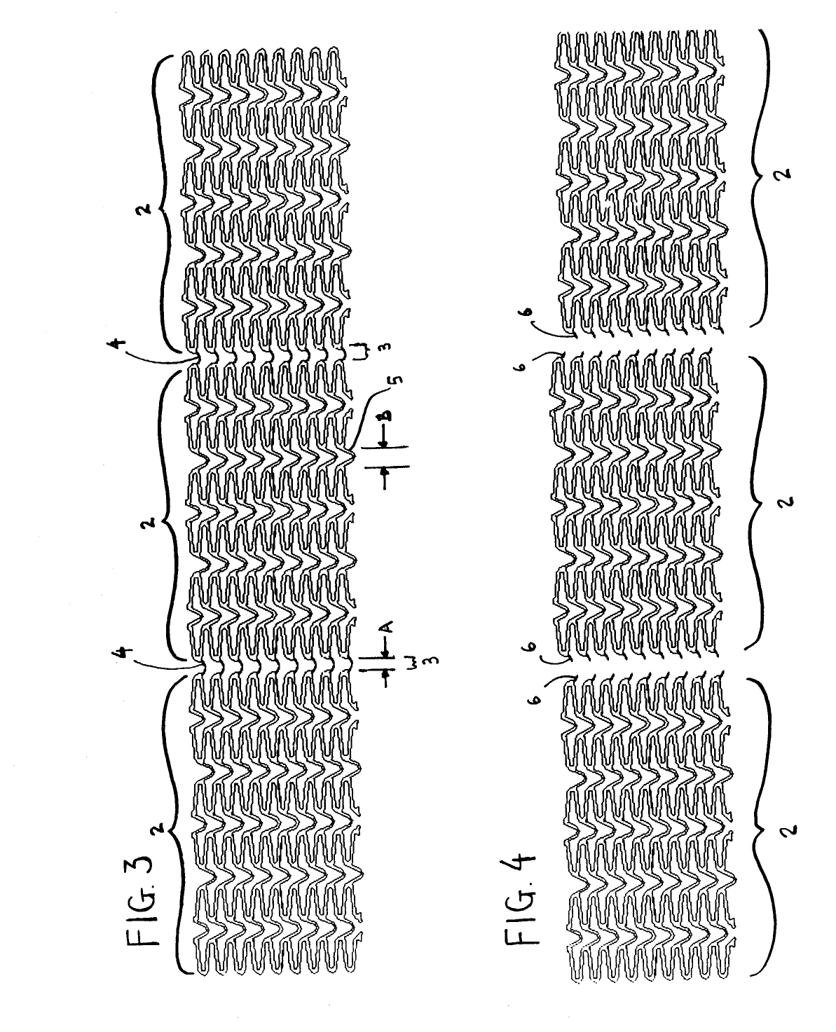

FIG. 4 shows a flat layout of the stent pattern of FIG. 3 after detachment has occurred at the designated detachment zones 3. As shown in FIG. 4, the stent after detachment comprises a series of separated and independent stent segments 2. As also seen in FIG. 4, because the designated detachment struts 4 were short, the length of the free ends 6 after separation is kept to a minimum.

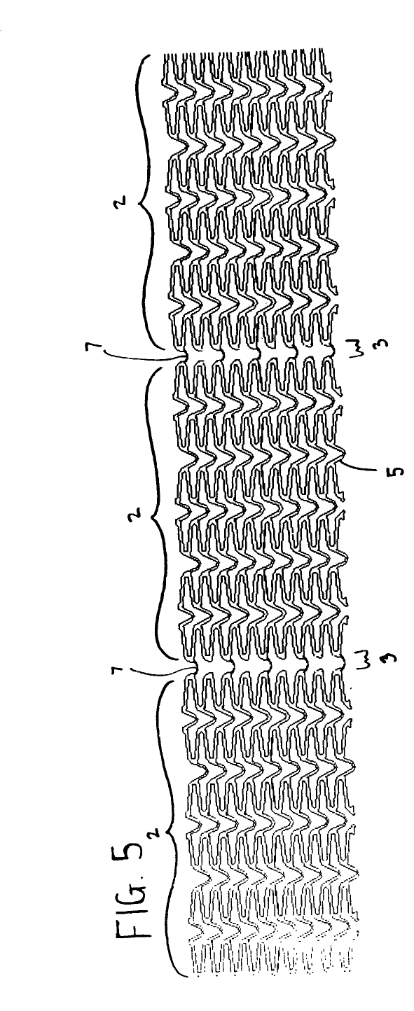

FIG. 5 shows an alternative design in which, in the designated detachment zones 3, the stent is provided with a lower number of components 7 around the circumference of the stent. In the embodiment shown in FIG. 5, each designated detachment zone 3 has five designated detachment struts 7 around the circumference of the stent. By comparison, the stent has nine of the components labeled as component 5 in a band of such components within the stent segments 2. Of course, different numbers of designated detachment struts and stent segment components may be used, without departing from the general concept of the invention.

The designated detachment struts 7 are configured such that they detach under the loads they bear on account of the stress placed on the stent after implantation. As shown in FIG. 5, the designated detachment struts 7 may also have a reduced cross-sectional area. Also, as with the designated detachment struts in other embodiments, the designated detachment struts 7 may additionally be formed of weaker material, or the designated detachment struts 7 or zones 3 may be treated to make the material weaker after production of the stent.

The embodiments described herein are examples only, as other variations are within the scope of the invention as defined by the appended claims.

What is claimed is:

1. A stent for implantation in a vessel, wherein the stent comprises:
(a) a plurality of stent segments; and
(b) a plurality of means to permit said adjacent stent segments to separate from each other in response to physiological stress placed on said means such that said separation occurs after a period of time after implantation of the stent in the vessel, the period of time being sufficient to permit neointima formation around the stent in an amount sufficient to secure said plurality of stent segments with respect to the vessel, said means located between and detachably connecting adjacent stent segments of said plurality of stent segments.

2. A stent as claimed in claim 1, wherein said means to permit comprise designated detachment struts.

3. A stent as claimed in claim 2, wherein the cross-sectional area of the designated detachment struts are sufficiently low so that the designated detachment struts will separate preferentially under stress placed on the stent after implantation.

4. A stent as claimed in claim 2, wherein the designated detachment struts are made of a material that is sufficiently weaker than elsewhere in the stent so that the designated detachment struts will separate preferentially under stress placed on the stent after implantation.

5. A stent as claimed in claim 2, wherein the designated detachment struts have a cross-sectional area that is less than the cross-sectional area of a component within one of said stent segments.

6. A stent as claimed in claim 5, wherein the designated detachment struts are also made of a material that is weaker than the material of a component within one of said stent segments.

7. A stent as claimed in claim 2, wherein the designated detachment struts are made of a material that is weaker than the material of a component within one of said stent segments.

8. A stent as claimed in claim 2, wherein the designated detachment struts are in a designated detachment zone of the stent, and wherein the number of designated detachment struts in said designated detachment zone is less than the number of struts that traverse a plane that crosses one of said stent segments perpendicular to an axis of the stent segment.

9. A stent as claimed in claim 8, wherein the at least one designated detachment strut is also made of a material that is weaker than the material of a component within one of said stent segments.

10. A stent as claimed in claim 9, wherein at least one designated detachment strut is made of a material that is weaker than the material of a component within one of said stent segments.

11. A method of employing a stent comprising at least two stent segments and a plurality of designated detachment struts between the at least two stent segments comprising:
a. forming each of the designated detachment struts such that, in response to physiological stress placed on the designated detachment struts said struts will separate after a period of time after implantation of the stent in a vessel, the period of time being sufficient to permit neointima formation around the stent in an amount sufficient to secure said plurality of stent segments with respect to the vessel;
b. disposing the stent within a vessel;
c. permitting neointima formation around the stent in an amount sufficient to secure said plurality of stent segments with respect to the vessel.
d. separating the two stent segments from each other in response to physiological stress placed on the designated detachment struts after the neointima formation.

12. The method according to claim 11, wherein said step of forming comprises forming the at least one designated detachment strut of a material that is weaker than the material of a component within one of said stent segments.

13. The method according to claim 11, comprising disposing the designated detachment struts in a designated detachment zone of the stent, and selecting the number of designated detachment struts in the designated detachment zone to be fewer than the number of struts that traverse a plane that crosses one of said stent segments perpendicular to an axis of the stent segment.

14. The method according to claim 13, wherein said step of forming comprises making the designated detachment struts of a material that is weaker than the material of a component within one of said stent segments.

15. The method according to claim 12, wherein said step of forming comprises treating the designated detachment strut to make the material weaker after production of the stent.

16. The method according to claim 12, wherein said step of forming comprises making the designated detachment strut with a cross-sectional area that is less than the cross-sectional area of a component within one of said stent segments.

17. The method according to claim 12, wherein said stent is made from metal.

18. A method according to claim 1, wherein the stent is made from metal.
